(19) United States
(12) Patent Application Publication
(10) Pub. No.: US 2022/0115097 A1
(43) Pub. Date: Apr. 14, 2022

(54) SYSTEM AND METHODS FOR A CLOUD-BASED USER-INTERACTIVE PARTOGRAM

(71) Applicant: CERNER INNOVATION, INC., Kansas City, KS (US)

(72) Inventors: Namratha Bangre Prabhakara Rao, Bengaluru (IN); Deepak Gupta, Bengaluru (IN); Chinnu Binish, Bengaluru (IN); HARSHAGIRI RAMAPRASANNA KUMAR, Bengaluru (IN); Kate Lynn Hawkins Spahr, Leawood, KS (US)

(21) Appl. No.: 17/213,708

(22) Filed: Mar. 26, 2021

Related U.S. Application Data
(60) Provisional application No. 63/090,443, filed on Oct. 12, 2020.

(51) Int. Cl. G16H 10/60; H04L 29/06; H04L 29/08

(52) U.S. Cl. CPC G16H 10/60; H04L 67/40; H04L 67/1097; H04L 67/42

(57) ABSTRACT

Methods, systems, and computer-readable media are disclosed herein for generating a user-interactive partogram in a graphical user interface using cloud deployment and multi-application integration.

### CROSS REFERENCE TO RELATED APPLICATION

This application is a Nonprovisional that claims the benefit of priority to U.S. Provisional No. 63/090,443, filed on 12 Oct. 2020, the entirely of which is incorporated by reference herein.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

In brief and at a high level, this disclosure describes, among other things, methods, systems, and computer-readable media for a cloud-based user-interactive partogram. In one aspect, a method is provided for facilitating a computerized partogram. In the method, a graphical user interface is generated that presents a user-interactive partogram, wherein a plurality of applications are integrated into the user-interactive partogram. A display of the user-interactive partogram in the graphical user interface is provided, wherein each of the plurality of applications are displayed in a corresponding portion of the graphical user interface. User input is received via the graphical user interface, and in response an electronic record is modified from within the user-interactive partogram itself. The user-interactive partogram being displayed is updated in the graphical user interface based on the electronic record being modified.

In another aspect, a system is provided. The system includes a cloud-based partogram platform and a plurality of applications that are integrated within the cloud-based partogram platform. The plurality of applications include a graphical user interface component for generating and causing display of a user-interactive partogram and a documentation component for direct charting, within the cloud-based partogram platform, information to a patient's electronic record. The system further includes an overview component, a labor curve component, a contractions component, a maternal vital signs component, a maternal labor assessment component, a medication administration record component, a fetal heart rate component, and a fetal assessment component.

In yet another aspect, one more non-transitory computer-readable media having computer-executable instructions embodied thereon are provided that, when executed, perform a method. In accordance with the media, a graphical user interface is generated to display a cloud-based user-interactive partogram having a plurality of applications that are integrated into the cloud-based user-interactive partogram, wherein the graphical user interface generated is specific to a first patient. The plurality of applications of the cloud-based user-interactive partogram are concurrently displayed in the graphical user interface, wherein each of the plurality of applications are displayed in a corresponding portion of the graphical user interface, and wherein information specific to the first patient is provided by each of the plurality of applications is being updated in near real-time as displayed in the corresponding portion of the graphical user interface. A request is received via the graphical user interface and, in response, an electronic record corresponding to the first patient is directly modified from within the graphical user interface having the cloud-based user-interactive partogram. The cloud-based user-interactive partogram is updated and displayed in the graphical user interface based on the electronic record being modified.

### BRIEF DESCRIPTION OF THE DRAWINGS

Aspects are described in detail below with reference to the attached drawings figures, wherein:

FIG. 1 is a block diagram of a system, in accordance with aspects of the present disclosure;

FIG. 2 is a diagram of a cloud-based architecture for the system of FIG. 1, in accordance with aspects of the present disclosure;

FIG. 3 is a diagram of one or more components for the system of FIG. 1, in accordance with aspects of the present disclosure;

FIG. 4 is another diagram of one or more components for the system of FIG. 1, in accordance with aspects of the present disclosure;

FIG. 5 is an overview portion of a graphical user interface of an unpopulated overview display, in accordance with aspects of the present disclosure;

FIG. 6 is an overview portion of a graphical user interface of a populated overview display, in accordance with aspects of the present disclosure;

FIG. 7 is the overview portion of the graphical user interface of FIG. 5 partially overlaid with a detailed information pop-up window, in accordance with aspects of the present disclosure;

FIG. 8 depicts a graphical user interface partially overlaid with a direct entry pop-up window, in accordance with aspects of the present disclosure;

FIG. 9 depicts a graphical user interface with a populated labor curve portion, in accordance with aspects of the present disclosure;

FIG. 10 depicts the graphical user interface of FIG. 9 with a detailed information pop-up window of the labor curve, in accordance with aspects of the present disclosure;

FIG. 11 depicts the graphical user interface of FIG. 9 with a detailed information pop-up window of a timeline, in accordance with aspects of the present disclosure;

FIG. 12 depicts a vital signs portion of the graphical user interface of FIG. 9, in accordance with aspects of the present disclosure;

FIG. 13 depicts a medication administrations record portion of the graphical user interface of FIG. 9, in accordance with aspects of the present disclosure;

FIG. 14 depicts a maternal labor assessments portion of the graphical user interface of FIG. 9, in accordance with aspects of the present disclosure;

FIG. 15 depicts a contractions portion of the graphical user interface of FIG. 9, in accordance with aspects of the present disclosure;

FIG. 16 depicts a configuration window, in accordance with aspects of the present disclosure;

FIGS. 17A-B depicts a flow diagram for populating one or more of the graphical user interfaces, in accordance with aspects of the present disclosure;

FIGS. 18 and 19 depict various methods in accordance with aspects of the present disclosure; and

FIG. 20 depicts a computing environment suitable for implements of the aspects of the present disclosure.

### DETAILED DESCRIPTION

The subject matter of the present invention is being described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Terms should not be interpreted as implying any particular order among or between various steps herein disclosed unless and except when the order of individual steps is explicitly described. As such, although the terms “step” and/or “block” can be used herein to connote different elements of systems and/or methods, the terms should not be interpreted as implying any particular order and/or dependencies among or between various components and/or steps herein disclosed unless and except when the order of individual steps is explicitly described. The present disclosure will now be described more fully herein with reference to the accompanying drawings, which may not be drawn to scale and which are not to be construed as limiting. Indeed, the present invention can be embodied in many different forms and should not be construed as limited to the aspects set forth herein. Further, it will be apparent from this Detailed Description that the technological solutions disclosed herein are only a portion of those provided by the present invention. As such, the technological problems, solutions, advances, and improvements expressly referenced and explained herein should not be construed in a way that would limit the benefits, improvements, and/or practical application of the discussed aspects of the present invention.

Aspects herein provide methods, systems, and computer-readable media for a user-interactive cloud-based partogram platform that integrates a plurality of diverse maternal and/or fetal monitoring tools and services into a common graphical user interface. Further, the user-interactive cloud-based partogram platform provides direct entry to medical records from within a common graphical user interface itself. The cloud-based user-interactive partogram provided enables increasingly accurate labor progression charting, which, in turn, facilitates earlier intervention opportunities to identify deviations from optimal and/or established guidelines for labor progression. Earlier and accurate deviation determinations allow for clinical intervention that increase positive material and fetal outcomes.

The terms “partograph” and “partogram” are used interchangeably herein, and the use of one term over another is not intended to indicate any difference between the two and is not intended to impart any particular limitations, unless expressly stated otherwise. As used herein, a “partogram” or “partograph” refers to a graphical representation of maternal and fetal data that is/has been recorded to document specific information concurrently with and/or during labor and delivery procedures. The term “platform” generally refers to a computer platform and/or software platform that can receive data and data streams from multiple, diverse, remote, and/or computer systems, devices (e.g., patient monitoring devices, mobile devices), and/or users (e.g., input to electronic medical records, medical order placement). The term “container” refers, generally, to an abstraction in the application layer for running a service, application, and/or virtual machine (e.g., an engine).

Existing systems and technologies rely on applications that use a reporting layer that operates on top of a read-only functionality, which is a configuration and arrangement that does not allow a user to access and enter new data and information directly into an electronic record from within the application.

In an improvement over existing systems and technologies, the cloud-implemented partogram platform herein facilitates the documentation and entry of new medical information into the electronic medical record of a patient using a graphical user interface, all without requiring a user to navigate to a new graphical user interface or to navigate to another application. In the aspects described hereinafter, Representational State Transfer-type services are used to expose a plurality of cloud-enabled and cloud-deployed Application Programming Interfaces (APIs) in order to enable a user to access and write directly to the electronic records from within a single application that integrates the plurality of APIs. As such, aspects of a system and method provided for herein include a configuration and arrangement of components that provide an improvement over existing systems and technologies by generating a new functionality.

Additionally, each time that information is written to the electronic records from within the single application that integrates the plurality of APIs, the cloud-implemented partogram platform of the system and method discussed herein automatically, and in near real-time, updates a graphical user interface that presents each of the plurality of APIs, all without requiring refreshing and/or re-loading of the single application. In contrast, the use of a reporting layer operating on top of a read-only functionality as found in existing systems and technologies, required refreshing and/or re-loading of a partogram application each time information was indirectly entered by backing out of a partogram view and navigating to another screen and/or to an external application. As such, aspects of a system and method provided for herein provide another improvement over existing systems and technologies because the new functionality that enables direct entry to an electronic record from within the application does not require a user to back out from a partogram view, navigate to another screen and/or to an external application to enter information, and further, does not require a refresh and/or a reload for each instance when new information entered.

Further, the cloud-implemented partogram platform of the system and method discussed herein provides a single application that integrates all of the plurality of APIs into one configurable, continuous, and common graphical user interface. Existing systems and technologies could not provide a common graphical user interface for the multiple APIs due to software and/or hardware incompatibilities that prevented integration of multiple APIs in a configuration involving a reporting layer operating on top of a read-only functionality.

In addition, because the user-interactive partogram platform is deployed within a cloud network, a plurality of remote users and distributed devices can concurrently access and utilize the common graphic user interface of the partogram platform for multiple patients.

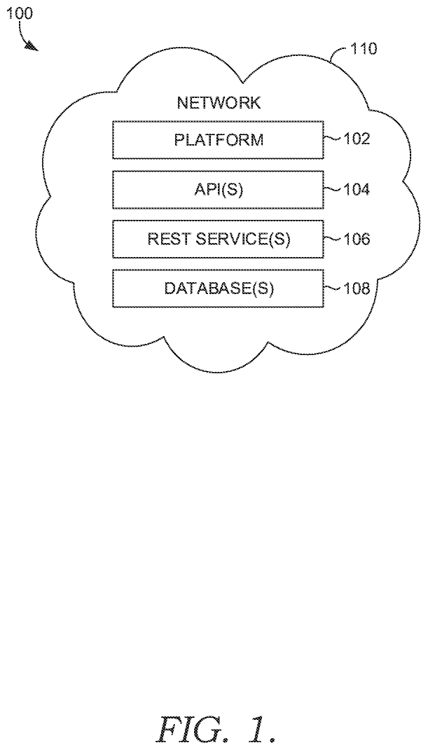

Beginning with FIG. 1, a block diagram of a system 100 for running and hosting a cloud-based partogram platform is provided, in accordance with aspects of the present disclosure. In FIG. 1, the system 100 operates within a network 110. In some aspects, the network 110 is a “cloud” type of network. In aspects, the system 100 includes a platform 102, one or more Application Programming Interfaces (APIs) 104, one or more Representational State Transfer (REST) service(s) 106, and/or one or more database(s) 108. The system 100 is configured to host and provide access to a cloud-deployed and cloud-accessible partogram platform that integrates a plurality of components (e.g., tools, services, applications, APIs). In aspects, the platform 102 is a partogram platform. The one or more APIs 104 can be components integrated into the cloud-based partogram platform, in some aspects.

The one or more REST service(s) 106 can be used to expose the one or more APIs 104 that are cloud-enabled and cloud-deployed, in some aspects. By using the one or more REST service(s) 106 to expose the one or more APIs in the cloud, the APIs can write directly to the one or more database(s) 108, in aspects, which was not feasible or possible in prior systems and prior technologies. The one or more database(s) 108 may include one or more types and/or any combination of different types of cloud storage services. For example, cloud storage services can include object storage, file storage, and/or block storage.

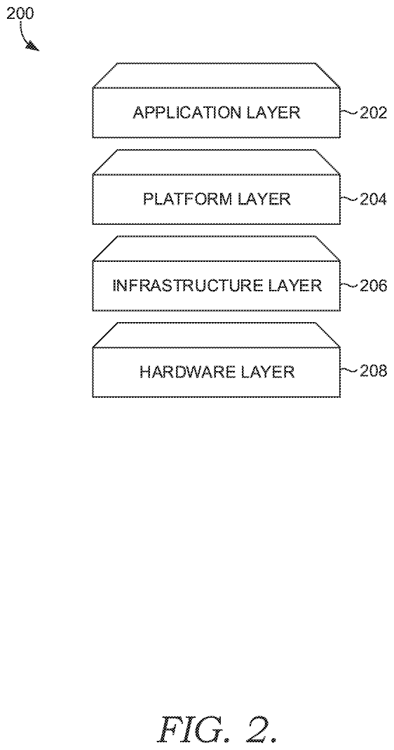

FIG. 2 is a diagram of a cloud-based architecture 200 suitable for supporting and hosting the system 100 of FIG. 1, in accordance with aspects of the present disclosure. In some aspects, the cloud-based architecture 200 can include one or more layers in a protocol stack. In various aspects, the protocol stack may include one or more layers in any combination of different layer types, such as, for example, a Software-as-a-Service (SaaS), Platform-as-a-Service (PaaS), and/or Infrastructure-as-a-Service (IaaS). The one or more layers can include an application layer 202, a platform layer 204, an infrastructure layer 206, and/or a hardware layer 208, in some aspects.

The application layer 202 can host software, applications, and/or one or more APIs, such as the one or more APIs 104 of the system 100 of FIG. 1. The platform layer 204 can, in some aspects, host a platform, such as the platform 102 of the system 100 of FIG. 1. For example, the platform layer 204 can host software frameworks (e.g., Java, Python) as well as storage (e.g., structure data bases). The infrastructure layer 206 can host and run one or more virtual machines, in various aspects. The hardware layer 208 can correspond to physical memory (e.g., disk) and processor (e.g., computer processing unit (CPUs)), in various aspects.

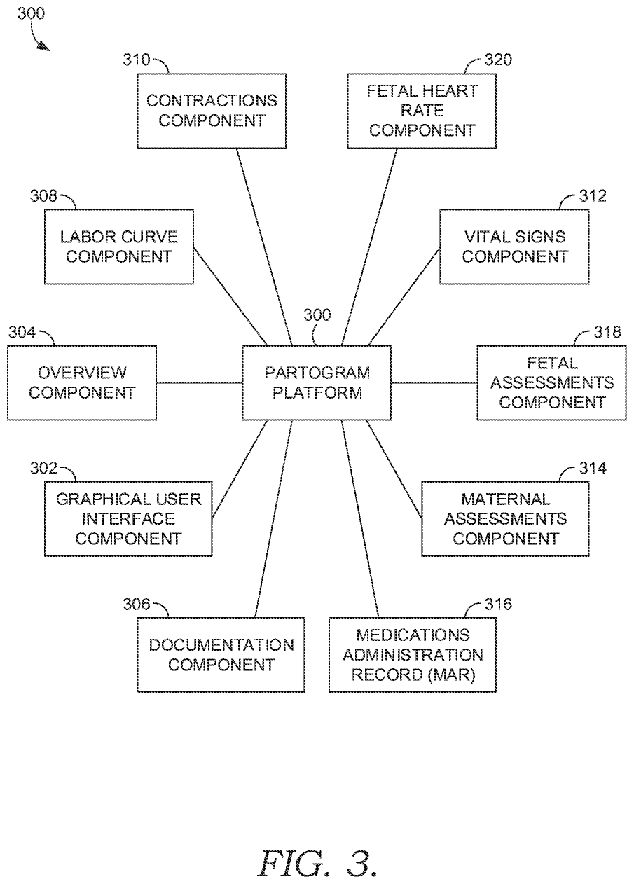

Continuing, FIG. 3 is a diagram of one or more components that are integrated into the cloud-based partogram platform 300 that is provided by the system 100 of FIG. 1, in accordance with aspects of the present disclosure. Each of the one or more components integrated into the cloud-based partogram platform may correspond to software, hardware, and/or APIs, such as the APIs 104 of FIG. 1, in some aspects. The cloud-based partogram platform may include one or more components, such as, for example, any combination of a graphical user interface component 302, an overview component 304, a documentation component 306, a labor curve component 308, a contractions component 310, a vital signs component 312, a maternal assessments component 314, a medications administration record component 316 (“MAR”), a fetal assessments component 318, and a fetal heart rate component 320.

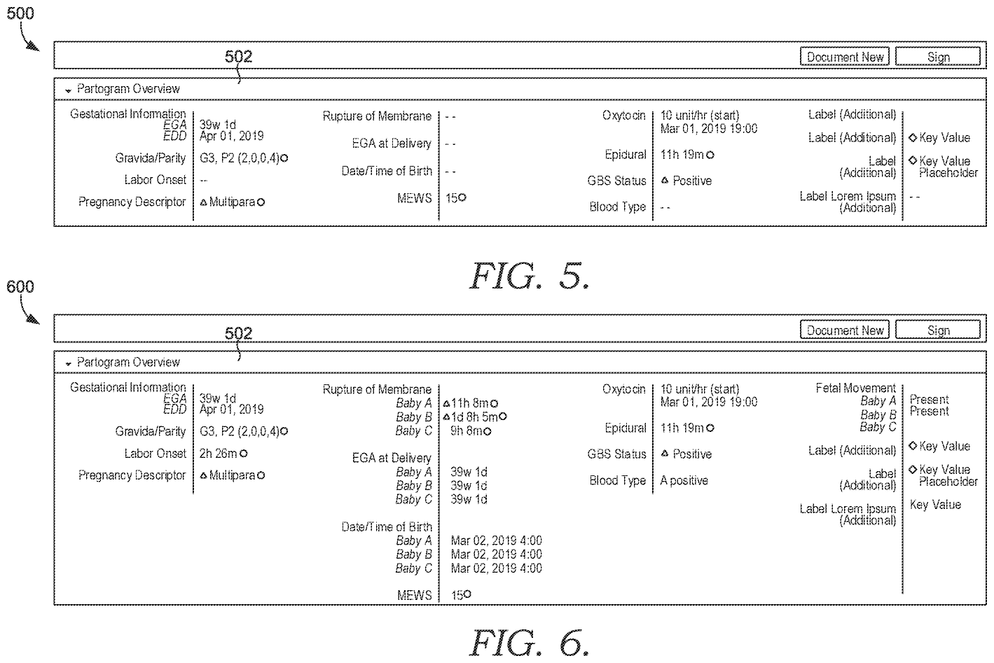

The graphical user interface component 302 can generate and cause the display of a common graphical user interface having a user-interactive partogram that integrates all of the plurality of APIs of the cloud-based user-interactive partogram platform. The common graphical user interface generated by the graphical user interface component 302 can be displayed as a continuous graphical user interface which a user can scroll through to reach discrete portions that include information for each of a plurality of APIs, in various aspects. The common graphical user interface generated by the graphical user interface component 302 can also be configurable, such that each of the discrete portions for the one or more APIs can be collapsed or expanded within the common graphical user interface, by user inputs and user interactions with selectable icons, buttons, objects, and the like displayed, in some aspects. As used herein, the term “common” refers to the characteristics having information from multiple APIs shown in one continuous graphical user interface, meaning that the user does not need to navigate to a new screen or window in order to access information for all of the APIs, and/or to access functionalities of the APIs themselves. Further, the graphical user interface component 302 may receive information from the APIs, for example, and, in response, the graphical user interface component 302 may update one or more portions of the user-interactive partogram in the graphical user interface in near real-time. In one example, the user-interactive partogram may be updated, as presented in the graphical user interface, based on an electronic record being modified through another API. Further, as discussed herein, FIGS. 5-16 provide one or more portions of the common graphical user interface, with windows and menus for displaying details, entering information, and/or configuring the partogram.

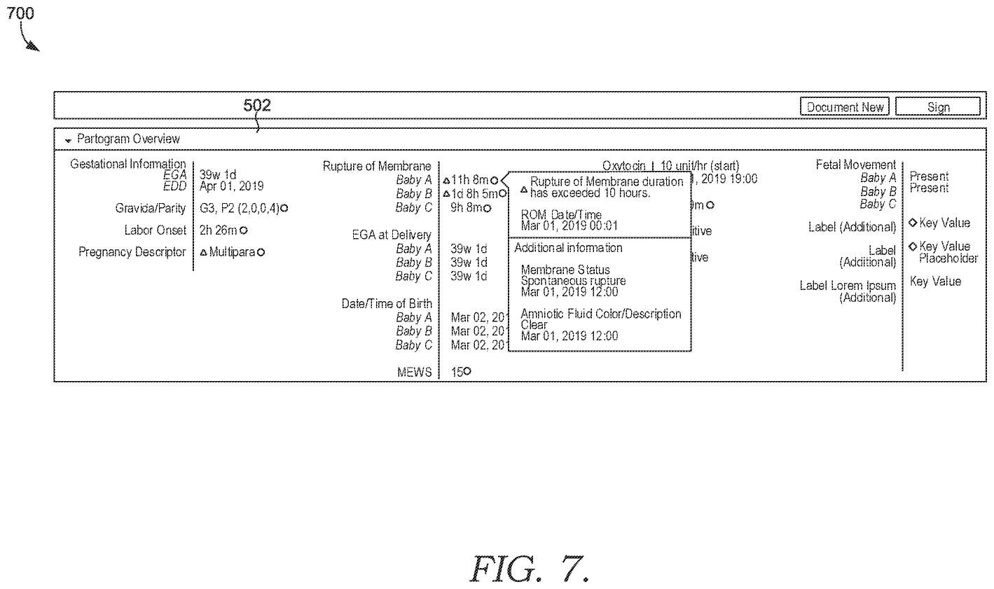

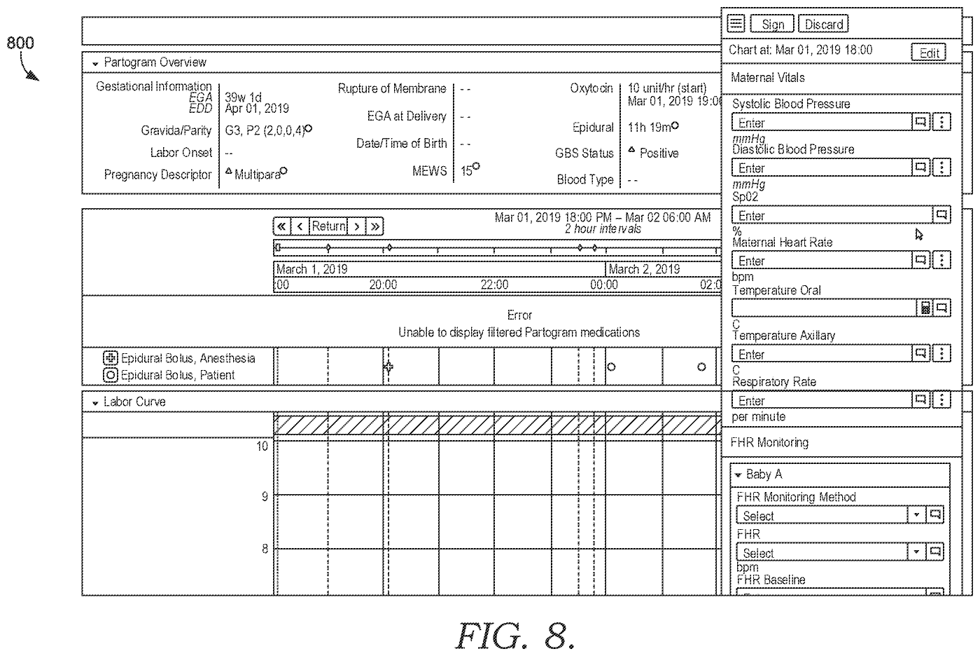

The overview component 304 may be a software service and/or application that aggregates information about a patient, for example, which may be displayed in a designated portion of a user-interactive partogram in a graphical user interface. For example, FIG. 5 depicts a graphical user interface 500 having an overview portion 502 that is unpopulated, whereas FIG. 6 depicts a graphical user interface 600 having the overview portion 502 that has been populated with patient-specific information, in accordance with aspects of the present disclosure. The overview portion 502 may be automatically populated with patient-specific information obtained by user entry and/or retrieved from a patient-specific electronic medical record in response to input of an onset date and time for labor of the patient, in an aspect. In some aspects, a user can interact with the overview portion 502 of the common graphical user interface of the partogram platform by using a pointer, cursor, or other selection indicator to hover, click, or otherwise select an information icon from the overview portion 502, which triggers a pop-up window that displays additional details about a patient. FIG. 7 depicts the overview portion 502 partially overlaid with a detailed information pop-up window, in accordance with aspects of the present disclosure.

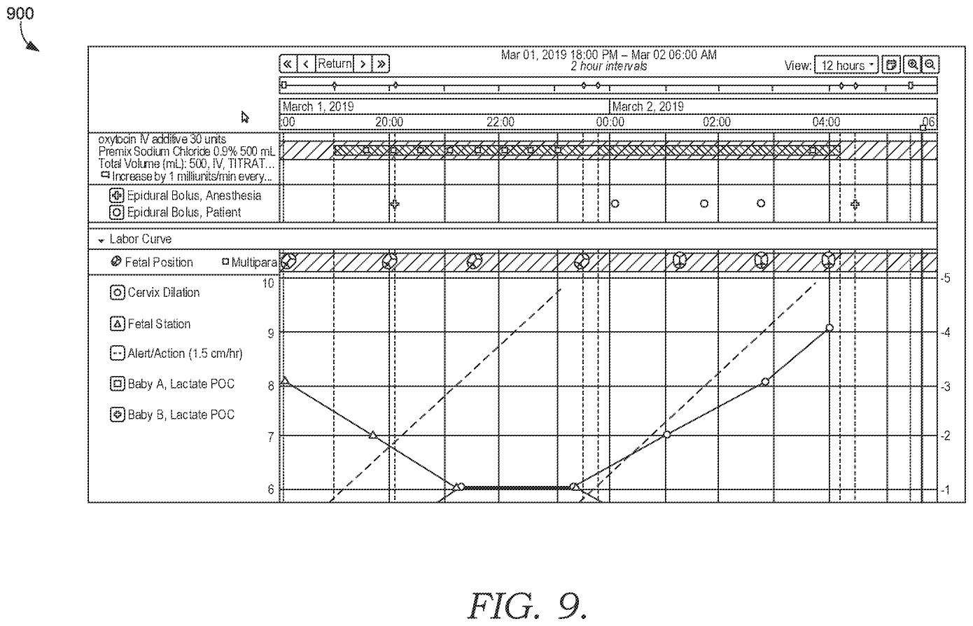

In various aspects, the documentation component 306 provides for the direct entry of new information, documentation, and/or data directly into an electronic medical record of a specific patient for which an instance of the partogram corresponds. In aspects, user input may be received and used by the documentation component 306 to modify and/or update, from within the graphical user interface having the user-interactive partogram, an electronic record in response to the user input. For example, a user selection of an icon, button, or other object in the graphical user interface having the user-interactive partogram, may enable entry to an electronic medical record through a pop-up window having one or more entry fields. FIG. 8 depicts a graphical user interface 800 partially overlaid with a direct entry pop-up window, in accordance with aspects of the present disclosure. In some aspects, the capability of the documentation component 306 to facilitate direct entry is supported by the one or more REST service(s) 106 exposing the documentation component 306 (e.g., an API), which is cloud-enabled and cloud-deployed, so that the documentation component 306 can receive input and directly write to the one or more database(s) 108, which store the electronic medical record of the specific patient for which the instance of the partogram corresponds.

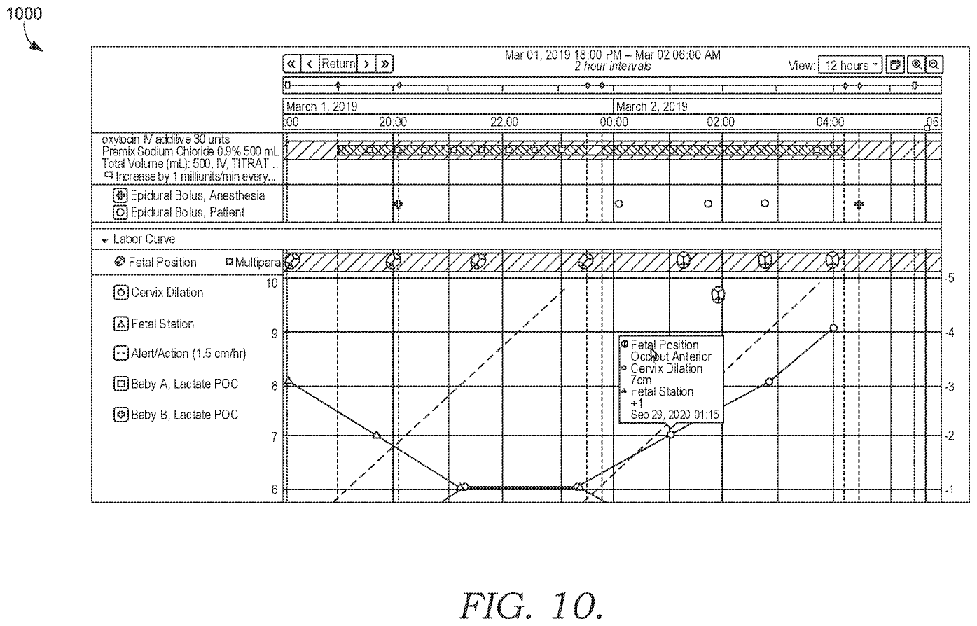

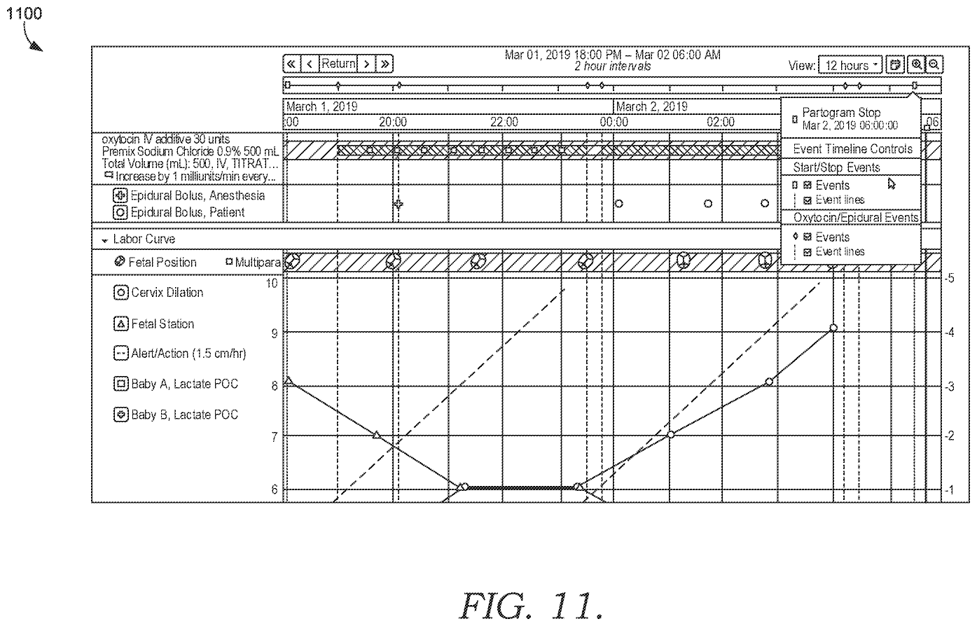

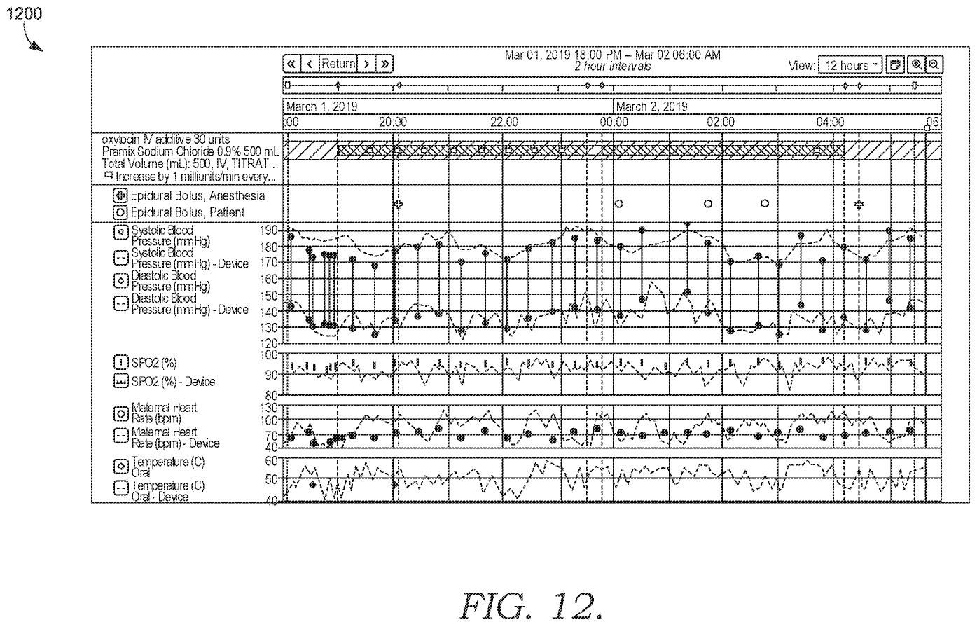

The labor curve component 308 may be a software service and/or application that graphs, in real-time, the progression of a patient's labor and delivery based on data received from, for example, maternal monitoring devices, manually input user information, and/or data from an electronic medical record (e.g., clinical notes). In one example, the labor curve component 308 may produce a linear-type of graph or other graph (e.g., change over time) representation of the patient's labor, for example, which may be displayed in a designated portion of the user-interactive partogram in the graphical user interface. FIG. 9 depicts a graphical user interface with a populated labor curve portion, in accordance with aspects of the present disclosure. FIG. 10 depicts the graphical user interface of FIG. 9 with a detailed information pop-up window of the labor curve, in accordance with aspects of the present disclosure. Further, FIG. 11 depicts the graphical user interface of FIG. 9 with a detailed information pop-up window of a timeline, in accordance with aspects of the present disclosure. As such, the labor curve component 308 may be used to present varied information on maternal labor progression through the graphical user interface.

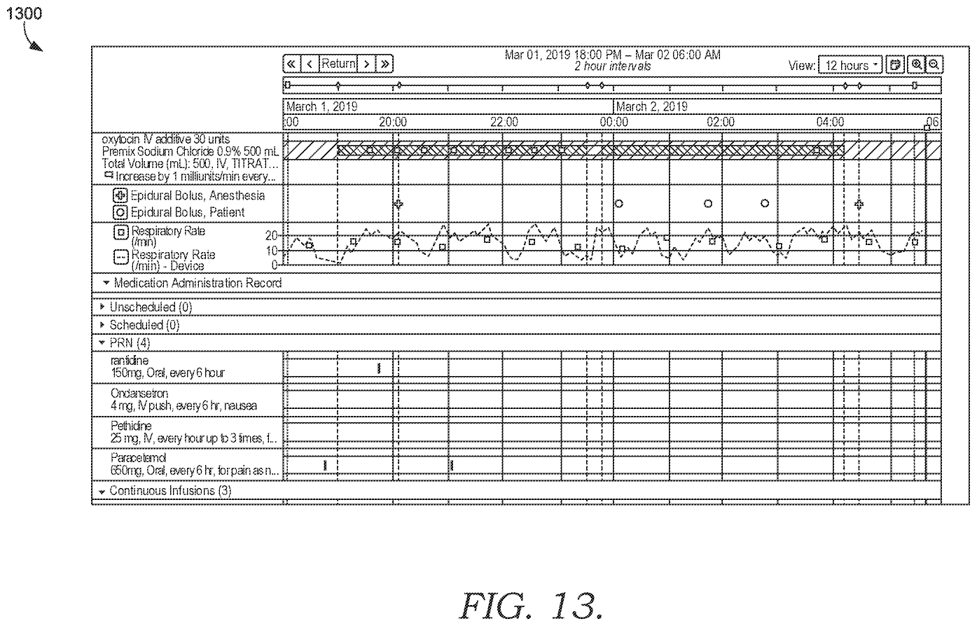

The contractions component 310 may be a software service and/or application that graphs, in real-time, the progression of a patient's labor contractions based on data received from, for example, maternal monitoring devices, manually input user information, and/or data from an electronic medical record (e.g., clinical notes). FIG. 12 depicts a contractions portion 1200 of the graphical user interface of FIG. 9, in accordance with aspects of the present disclosure. In the example of FIG. 12, the duration, strength, and frequency of contractions are presented in a unique bubble-graph in a portion of the user-interactive partogram that is designated for the presentation and display of data received from the contractions component 310.

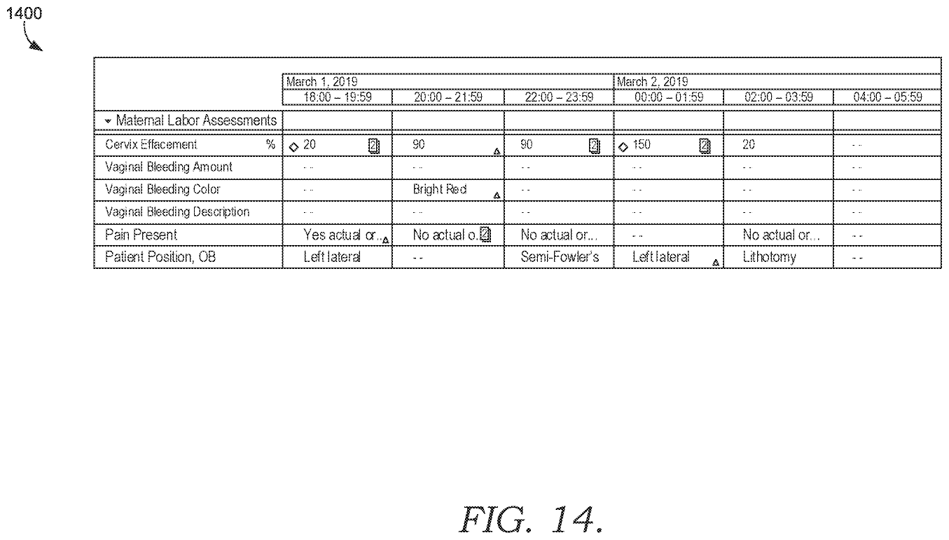

The vital signs component 312 may be a software service and/or application that measures and records, in real-time, maternal vital signs received from, for example, patient monitoring devices, manually input user information, and/or data from an electronic medical record (e.g., clinical notes) throughout labor and delivery. FIG. 13 depicts a vital signs portion 1300 of the graphical user interface of FIG. 9, in accordance with aspects of the present disclosure.

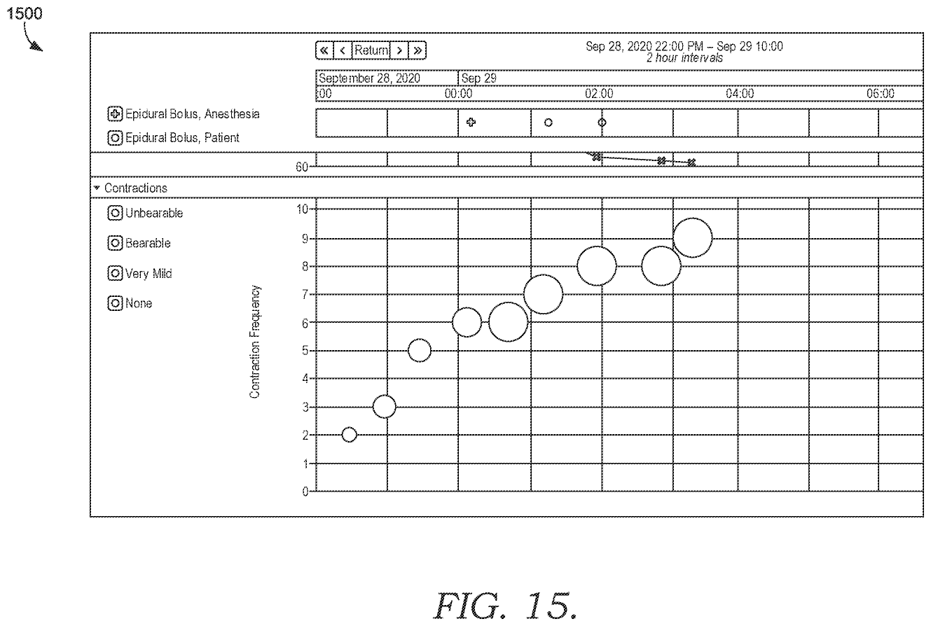

The maternal assessments component 314 may be a software service and/or application utilized by clinicians to receive, categorize, and store maternal assessments related to labor and delivery. FIG. 14 depicts a maternal labor assessments portion 1400 of the graphical user interface of FIG. 9, in accordance with aspects of the present disclosure.

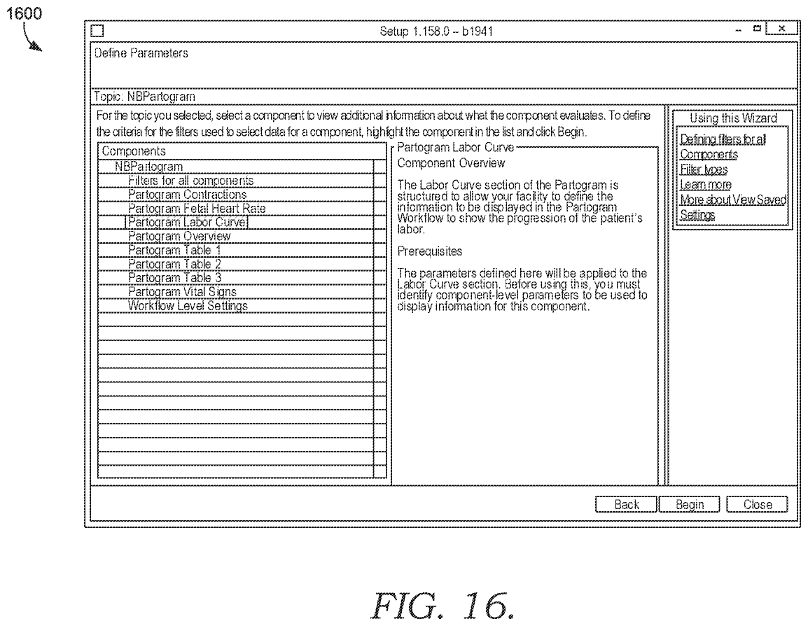

The MAR component 316 may be a software service and/or application utilized by clinicians to receive, categorize, and store maternal assessments related to labor and delivery. FIG. 15 depicts a medication administrations record portion 1500 of the graphical user interface of FIG. 9, in accordance with aspects of the present disclosure.

The fetal assessments component 318 and the fetal heart rate component 320 may be software services and/or applications that measure and record, in real-time, medical information of the fetus such as position within the uterus, a heart rate, for example, through patient monitoring devices, manually input user information, and/or data from an electronic medical record (e.g., clinical notes) throughout labor and delivery.

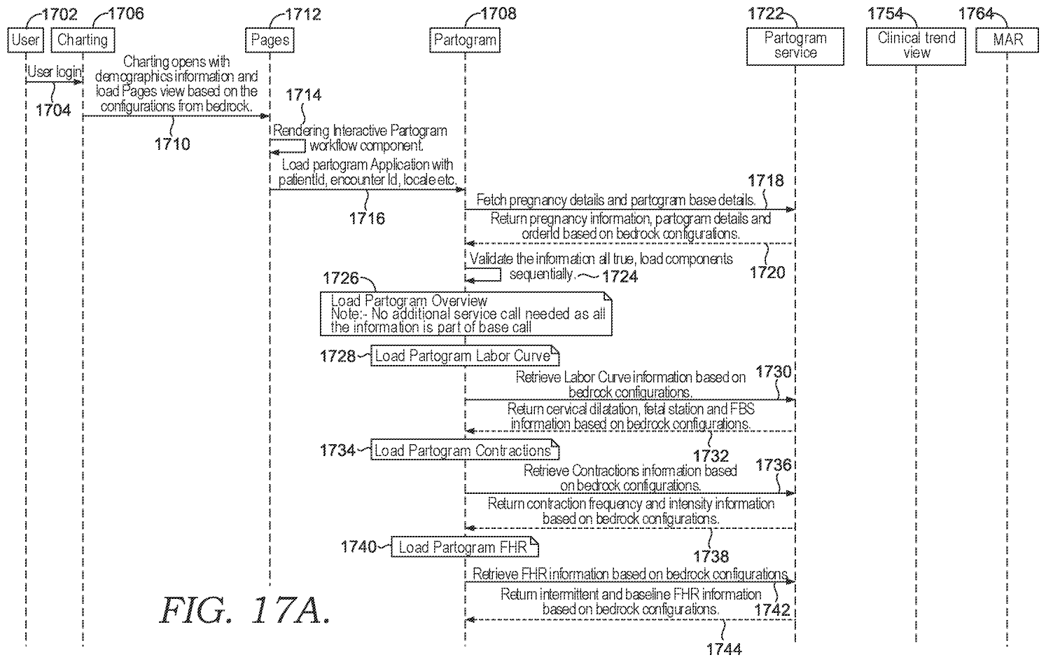

FIG. 16 depicts a configuration window 1600 for entering parameters to customize the partogram and the common graphical user interface, in accordance with aspects of the present disclosure.

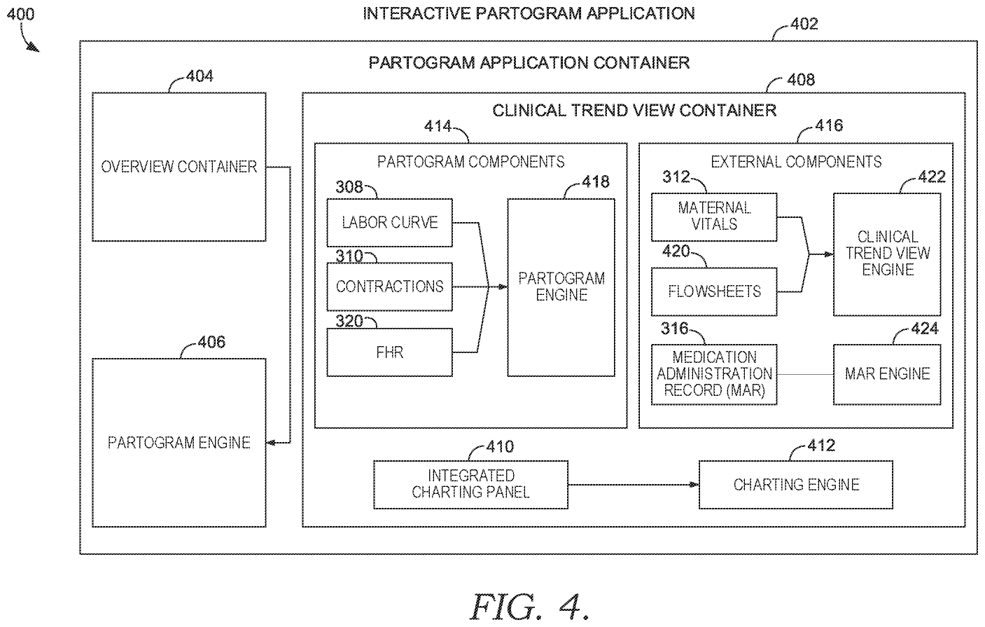

Having described the common graphical user interface and various components of the user-interactive cloud-based partogram platform that integrates a plurality of diverse maternal and/or fetal monitoring tools and services, FIG. 4 provides another diagram of one or more components of the system 100 of FIG. 1 that are arranged within a cloud-based architecture 200 of FIG. 2. In the example of FIG. 4, the user-interactive cloud-based partogram platform 400 comprises an application container 402, an overview container 404, a partogram engine 406, and a clinical trend container 408. The application container 402 operates to host applications, tools, and services provided through the user-interactive cloud-based partogram platform 400. The overview container 404 may include and/or host the overview component 304 discussed above, and as shown in FIGS. 5 and 6, in some aspects. The clinical trend container 408 and the overview container 404 can provide input to the partogram engine 406, in various aspects.

The clinical trend container 408 can include an integrated charting panel 410 and a charting engine 412. The integrated charting panel 410 and the charting engine 412 can facilitate the direct entry charting discussed above, with regard to the documentation component 306 and the graphical user interface partially overlaid with a direct entry pop-up window, as shown in FIG. 8.

The clinical trend container 408 can also include one or more partogram components 414 and one or more external components 416. The one or more partogram components 414 can include the labor curve component 308, the contractions component 310, and the fetal heart rate component 320, in some aspects, as previously described above. In one aspect, the labor curve component 308, the contractions component 310, and the fetal heart rate component 320 (“FHR”) provide real-time data and information to a clinical-trend container partogram engine 418. The one or more external components 416 can include the (maternal) vital signs component 312 and one or more flowsheets 420 detailed stages and information for labor progression. The vital signs component 312 and the one or more flowsheets 420 may provide input to a clinical trend view engine 422, in some aspects. In some aspects, the MAR component 316 provides input to a MAR engine 424. The clinical trend view engine 422, the MAR engine 424, and the clinical trend container partogram engine 418 may provide information used to generate and update the common graphical user interface and portions thereof.

Based on the description of the cloud-based architecture, the system, and the one or more components thereof, it will be understood by those of ordinary skill in the art that the depicted configurations and features are but just some examples that are suitable for the cloud-based architecture, the system, and the components and are not intended to limit the scope of use or functionality of the present invention. Similarly, the cloud-based architecture, the system, and the components should not be interpreted as imputing any dependency and/or any requirements with regard to each component and combination(s) of components illustrated in FIGS. 1-3. It will be appreciated by those of ordinary skill in the art that the configuration, locations, and arrangements of components illustrated in FIGS. 1-3 are just examples, as other methods, systems, hardware, software, components, and devices for establishing communication links between the cloud-based architecture, the system, and the components shown in FIGS. 1-3, may be utilized in implementations of the present invention. It will be understood to those of ordinary skill in the art that the components may be connected in various manners, hardwired or wireless, and may use intermediary components that have been omitted or not included in FIGS. 1-3 for simplicity's sake. As such, the absence of components from FIGS. 1-3 should be not be interpreted as limiting the present invention to exclude additional components and combination(s) of components. Moreover, though components are represented in FIGS. 1-3 as singular components, it will be appreciated that some aspects may include a plurality of devices and/or components such that FIGS. 1-3 should not be considered as limiting the number of a device or component.

Additionally, the operations, functions, and/or steps discussed above can correspond to a computer-implemented method. In one aspect, one or more non-transitory computer-readable media having computer-readable instructions or computer-readable program code portions embodied thereon, for execution via one or more processors, can be used to implement and/or perform the operations, functions, and/or steps discussed. For example, computer-readable instructions or computer-readable program code portions can specify a sequence of operations, functions, and/or steps, and/or can identify particular component(s) of a software and/or hardware for performing one or more operations, functions, and/or steps. The computer-readable instructions or computer-readable program code portions can correspond to an application and/or an application programming interface (API), in some aspects. Accordingly, as discussed, the operations, functions, and/or steps can be performed using software, hardware, component(s), and/or device(s) depicted in the example of FIGS. 1-3.

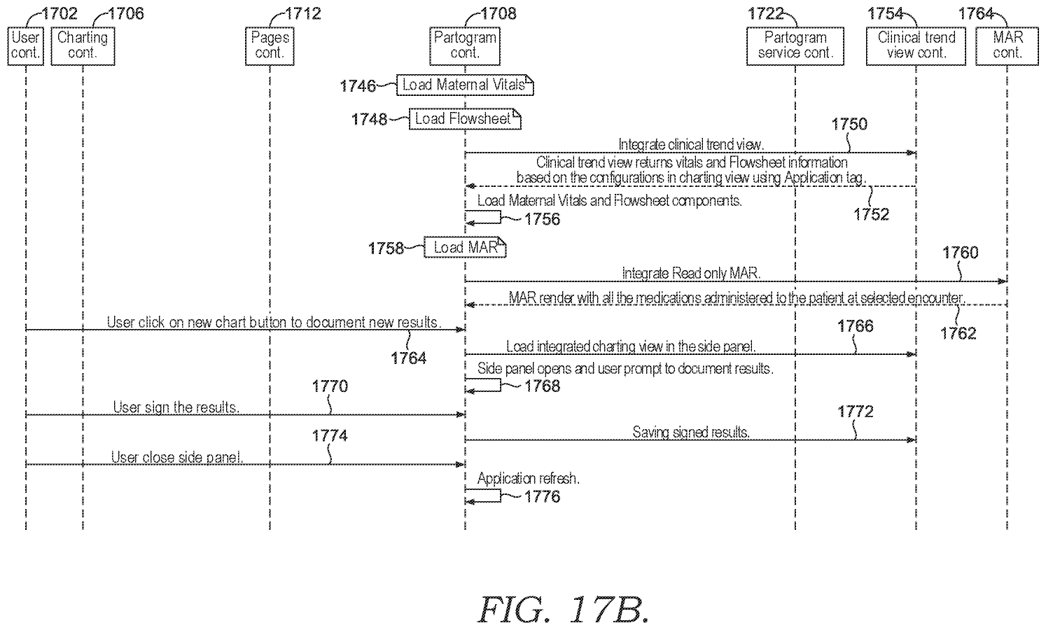

Looking now to FIGS. 17A-B, the figures depict a flow diagram 700 for populating one or more of the graphical user interfaces previously described, in accordance with aspects of the present disclosure. FIGS. 17A-B provide an example scenario showing the partogram platform in action, for example, by way of the components shown in FIG. 4 and as presented through the graphical user interfaces previously discussed herein.

In aspects, a user 1702 (i.e., via a user device) initiates a request 1704 to a charting application 1706, such as a user login request that specifies a unique identifier, password, code, and/or token. The charting application 1706 may utilize the request 1704 and information therein to authorize the user's access to the charting application and other applications, such as a partogram application 1708. The charting application 1706 may be initiated, launched, and/or “opened” based on authorization of the user 1702. The charting application 1706 may load with one or more demographics specific to a patient, for example. Additionally, the charting application 1706 may load a graphical user interface application with one or more configurations, for example, default configurations, user-specific configurations, patient-specific configurations (e.g., based on the patient-specific demographics), or a combination thereof. The charting application 1706 communicates 1710 information to a pages application 1712, wherein the pages application 1712 is an example of a graphical user interface application for accessing and/or interfacing with one or more backend or cloud-based applications. The information may cause the pages application 1712 to initiate, launch, and/or generate a rendering 1714 of one or more workflows of a partogram application 1708. For example, the information may specify that patient “Jane Doe” has a gender of female and has a positive “pregnancy” indicator, and further may include a request to launch the partogram application 1708 for patient Jane Doe. Once the pages application 1712 initiates, launches, and/or generates the rendering 1714 of one or more workflows of a partogram application 1708, the pages application 1712 may load 1716 graphical user interfaces (e.g., “views”) for the partogram application 1708. The view(s) of the partogram application 1708 may initially be default views populated with basic information, for example, the patient's identifier of Jane Doe, the patient's gender, the patient's physical location at a hospital, etc., as provided to the partogram application 1708 from the charting application 1706 (e.g., application or service acting as a source that feeds up-to-date information and data), via the pages application 1712 (e.g., graphical user interface rendering application).

Based on or in response to loading views of the partogram application 1708, the partogram application 1708 may fetch or request 1718 health information of the patient Jane Doe, for example, specific medical information regarding the pregnancy of Jane Doe such as pregnancy term (e.g., gestational week), age, race, number of prior pregnancies, and the like. Additionally, the partogram application 1708 may fetch, request, and/or retrieve default or base information, such as initial values to input or load for one or more services supported by the partogram application 1708 (e.g., labor curve, contractions, FHR). The partogram application 1708 may receive 1720 said information from a partogram service 1722, as well as default or initial “bedrock” configurations for the partogram application 1708. The partogram application 1708 may validate 1724 the information received. The partogram application 1708 may load 1726 an overview graphical user interface using the information and configurations discussed above (see FIGS. 5 and 6).

The partogram application 1708 may load one or more services subsequent to or in response to validation. For example, the partogram application 1708 may begin loading patient-specific information and/or populating views of the partogram application 1708 with the patient-specific information. In one example, a partogram labor curve service 1728 may be loaded by fetching or retrieving 1730 labor curve information which is communicated 1732 from the partogram service (see FIGS. 9 and 10). The partogram labor curve service 1728, generally, corresponds to the labor curve component 308 of FIG. 4. In another example, a partogram contractions service 1734 may be loaded by fetching or retrieving 1736 contractions information which is communicated 1738 from the partogram service (see FIG. 15). The partogram contractions service 1734, generally, corresponds to the contractions component 310 of FIG. 4. In yet another example, a partogram FHR service 1740 may be loaded by fetching or retrieving 1742 labor FHR information which is communicated 1744 from the partogram service. The partogram FHR service 1740, generally, corresponds to the FHR component 320 of FIG. 4. In various aspects, each service may be loaded concurrently, simultaneously, or sequentially in any order. Further, each service may load information that is specific to one patient, Jane Doe, while concurrently loading non-patient-specific information, such as standards of care or targets for contractions/labor curves and the like. As such, FIGS. 17A-B are not intended only as one example.

Continuing to FIG. 17B, a maternal vitals service 1746 is loaded and a flowsheet 1748 may be initiated for loading into the partogram application 1708. The flowsheet 1748 may request 1750 to integrate a clinical trend overview, which is returned 1752 from a clinical trend view service 1754. The clinical trend view service 1754 may, generally, correspond to and/or communicate with the clinical trend view engine 422 of FIG. 4. Based on the initiation and request(s), the maternal visits service 1746 and flowsheet 1748 may be fully loaded 1756 and populated into the partogram application 1708. The flowsheet 1748 may, generally, correspond to the flowsheet(s) component 420 of FIG. 4. The partogram application 1708 can further initiate loading of the MAR service 1758 by sending a request 1760 to integrate MAR information and receiving 1762 the requested MAR information for the patient Jane Doe from a MAR service 1764 (see FIG. 13). The MAR service 1758 may, generally, correspond to the MAR component 316 of FIG. 4, and further, may be supported by the MAR engine 424.

In the example shown in FIG. 17A, the user 1702 may provide an input that indicates a selection 1766 of a chart button in a graphical user interface of the partogram application 1708. In response to the selection 1766, the partogram application 1708 may load an integrated charting view in a side panel (see FIG. 8) of the graphical user interface, which is opened 1768 and loaded in the graphical user interface. In various aspects, an integrated charting view presented in a side panel of a graphical user interface may correspond to integrated charting panel 410 and/or may also be supported by the charting engine 412 of FIG. 4. The side panel, when opened and loaded, may include one or more prompts for user input and information regarding charting of medical information for patient Jane Doe. The user 1702 may provide an indication 1770 to the partogram application 1708, for example, an electronic signature of charted medication information for patient Jane Doe. The partogram application 1708 may communicate 1772 the charted medication information, the electronic signature, and/or additional information to the clinical trend view service 1754. In some aspects, the user 1702 may provide another indication 1774 to the partogram application 1708, for example, indicating the user 1702 is closing the side panel. Automatically and in response to the indication 1774 that the side panel is being closed, the partogram application 1708 may automatically refresh 1776 one or more, or all, of the services, in aspects. In other aspects, the partogram application 1708 may automatically refresh 1776 one or more, or all, of the services, automatically and in response to an indication of a user providing an electronic signature via the side panel, or another indication other than closing the side panel.

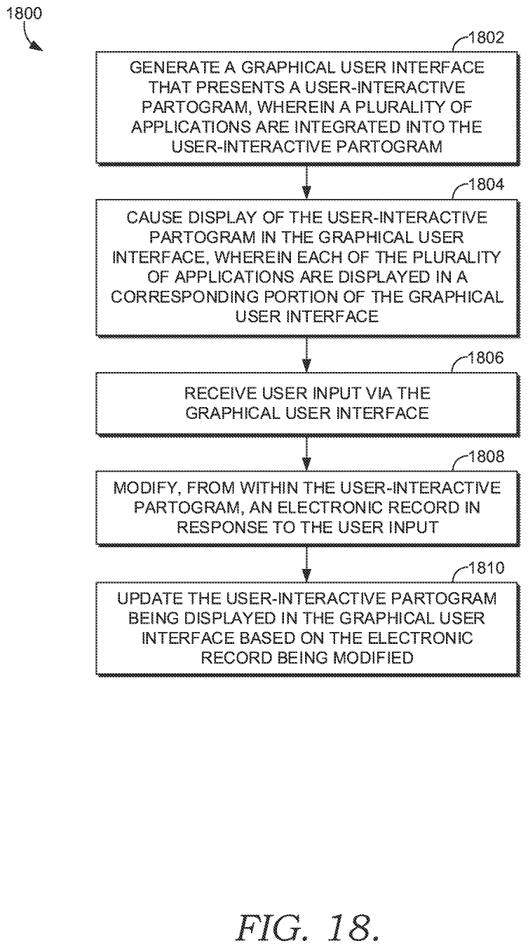

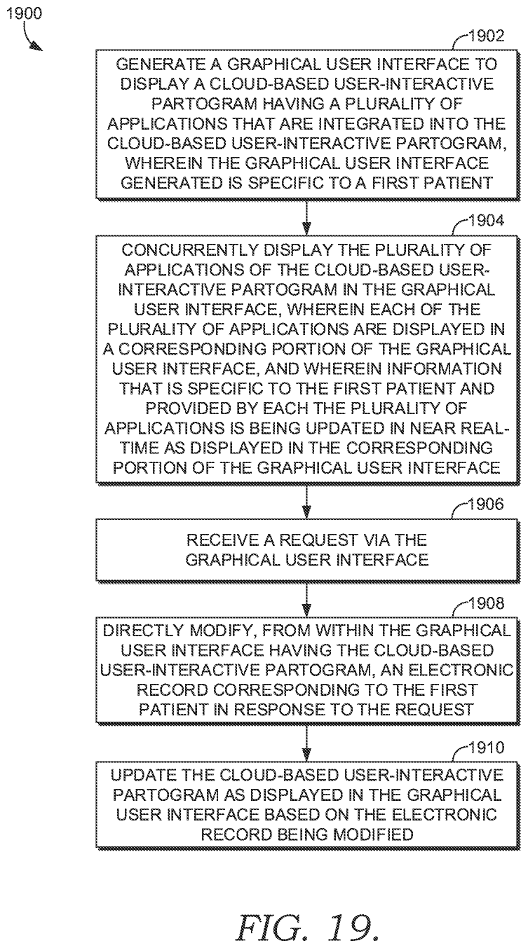

Having described an example scenario, FIGS. 18 and 19 are discussed hereinafter as providing example methods performed via the partogram platform for any iteration of scenarios. As such, the methods are discussed briefly for brevity, though it will be understood that the previous discussion and details described with regard to FIGS. 1-17 can be applicable to aspect of the methods of FIGS. 18 and 19. Additionally or alternatively, it will be understood that the methods discussed herein can be implemented or performed via the execution of computer-readable instructions stored on computer-readable media, by one or more processors.

FIG. 18 thus provides a method 1800 for producing a computerized partogram. At block 1802, a graphical user interface is generated that presents a user-interactive partogram, wherein a plurality of applications are integrated into the user-interactive partogram. In aspects, the user-interactive partogram is cloud-deployed and hosted in a platform layer. In such aspects, the plurality of applications are cloud-deployed and are hosted in an application layer. The user-interactive partogram is displayed in the graphical user interface, shown at block 1804, wherein each of the plurality of applications are displayed in a corresponding portion of the graphical user interface. User input is received via the graphical user interface at block 1806. At block 1808, an electronic record is modified from within the user-interactive partogram in response to the user input. A representational state transfer (REST) service exposes the plurality of integrated applications that are cloud-deployed to a database that stores the electronic record, in various aspects, wherein exposure of the plurality of applications by the REST service enables modification of the electronic record from within the user-interactive partogram. Then, the user-interactive partogram being displayed in the graphical user interface is updated based on the electronic record being modified, as shown at block 1810. Further, the user-interactive partogram is, generally, automatically updated as displayed in the graphical user interface in response to the modification, without further user input.

In one aspect, when the user input comprises a patient-specific value defining systolic blood pressure, diastolic blood pressure, SpO2, maternal heart rate, oral temperature, auxiliary temperature, respiratory rate, or a combination thereof, the electronic record is automatically modified by a charting engine to reflect the patient-specific value of the user input, without requiring navigation to a new screen.

FIG. 19 provides a method 1900 for producing a computerized partogram. At block 1902, a graphical user interface is generated to display a cloud-based user-interactive partogram having a plurality of applications that are integrated into the cloud-based user-interactive partogram, wherein the graphical user interface generated is specific to a first patient. The cloud-based user-interactive partogram may be run or executed within a cloud-based environment, in aspects. In various aspects, each of the plurality of applications of the cloud-based user-interactive partogram are automatically populated with information specific to an individual being monitored. The plurality of applications can include, for example, a graphical user interface component for generating and causing display of a user-interactive partogram, a documentation component for direct charting, within the cloud-based partogram platform, information to a patient's electronic record, an overview component, a labor curve component, a contractions component, a maternal vital signs component, a maternal labor assessment component, a medication administration record component, a fetal heart rate component, a fetal assessment component, or any combination thereof. In some aspects, a representational state transfer (REST) service is used to expose the cloud-deployed plurality of integrated applications to a database that stores a plurality of electronic records. In some aspects, the method 1900 includes receiving an input that specifies an onset date and time of labor for a patient. In such an aspect, and in response to receiving the onset date and time, information (described above) is automatically retrieved from the electronic record that is specific to the patient and populating the graphical user interface with the information retrieved.

The plurality of applications of the cloud-based user-interactive partogram are concurrently displayed, at block 1904, in the graphical user interface, wherein each of the plurality of applications are displayed in a corresponding portion of the graphical user interface, and information that is specific to the first patient and is being provided by each the plurality of applications is also being updated in near real-time as displayed in the corresponding portion of the graphical user interface. As such, the plurality of applications of the cloud-based user-interactive partogram being concurrently displayed in the graphical user interface can be continuously updating using near real-time information specific to an individual being monitored.

At block 1906, a request is received via the graphical user interface. From within the graphical user interface having the cloud-based user-interactive partogram, an electronic record corresponding to the first patient is automatically updated, edited, modified, and/or stored as changed in response to the request, shown at block 1908. In various aspects, via one or more REST services acting as an intermediary between a database storing the electronic record and the documentation application, direct modification of the electronic record is enabled through the documentation application in the cloud-based user-interactive partogram. At block 1910, the cloud-based user-interactive partogram is automatically updated as displayed in the graphical user interface based on and/or in response to the electronic record being modified.

In further aspects, a plurality of user input values are subsequently received. For each of the plurality of user input values that is subsequently received via the graphical user interface, the electronic record corresponding to the first patient is automatically and directly modified, from within the graphical user interface and in response to the input value. In such an aspect, the cloud-based user-interactive partogram is automatically updated as displayed in the graphical user interface based on the electronic record being modified.

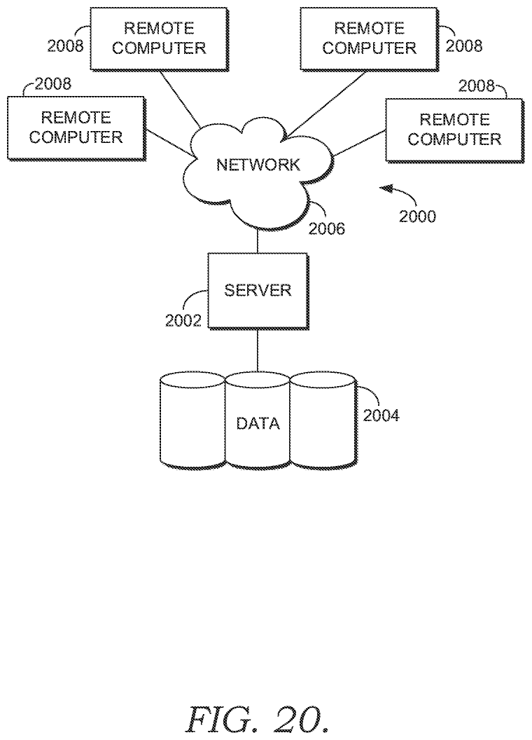

Turning to FIG. 20, a computing environment 2000 that is suitable for use in implementing aspects of the present invention is depicted. The computing environment 2000 is merely an example of one suitable computing environment and is not intended to suggest any limitation as to the scope of use or functionality of the invention. Neither should the computing environment 2000 be interpreted as having any dependency or requirement relating to any single component or combination of components illustrated therein. Generally, in aspects, the computing environment 2000 is a medical-information computing-system environment. However, this is just one example and the computing environment 2000 can be operational with other types, other kinds, or other-purpose computing system environments or configurations. Examples of computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

In aspects, the computing environment 2000 can be described in the general context of computer instructions, such as program modules, applications, and/or extensions, being read and executed by a computing device. Examples of computer instructions can include routines, programs, objects, components, and/or data structures that perform particular tasks or implement particular abstract data types. The aspects discussed herein can be practiced in centralized and/or distributed computing environments, i.e., where computer tasks are performed utilizing remote processing devices that are linked through a communications network, whether hardwired, wireless, or a combination thereof. In a distributed configuration, computer instructions might be stored or located in association with one or more local and/or remote computer storage media (e.g., memory storage devices). Accordingly, different portions of computer instructions for implementing the computer tool in the computing environment 2000 may be executed and run on different devices, whether local, remote, stationary, and/or mobile.

With continued reference to FIG. 20, the computing environment 2000 comprises a computing device 2002, shown in the example form of a server. Although illustrated as one component in FIG. 20, the present invention can utilize a plurality of local servers and/or remote servers in the computing environment 2000. The computing device 2002 can include components such as a processing unit, internal system memory, and a suitable system bus for coupling to various components, including electronic storage, memory, and the like, such as a data store, a database, and/or a database cluster. Example components of the computing device 2002 include a processing unit, internal system memory, and a suitable system bus for coupling various components, including a data store 2004, with the computing device 2002. An example system bus might be any of several types of bus structures, including a memory bus or memory controller, a peripheral bus, and a local bus, using any of a variety of bus architectures. Examples of bus architectures include Industry Standard Architecture (ISA) bus, Micro Channel Architecture (MCA) bus, Enhanced ISA (EISA) bus, Video Electronics Standards Association (VESA®) local bus, and Peripheral Component Interconnect (PCI) bus, also known as Mezzanine bus.

The computing device 2002 includes or has access to a variety of non-transitory computer-readable media. Computer-readable media can be any available media that is locally and/or remotely accessible by the computing device 2002, and includes volatile, nonvolatile, removable, and non-removable media. By way of example, and not limitation, computer-readable media can comprise computer storage media and communication media. Computer storage media includes volatile, nonvolatile, removable, and non-removable media, as implemented in any method or technology for storage of information, such as computer-readable instructions, data structures, program modules, or other data.

The computing device 2002 can include or can have access to computer-readable media. Computer-readable media can be any available media that can be accessed by computing device 2002, and includes volatile and nonvolatile media, as well as removable and non-removable media. By way of example, and not limitation, computer-readable media can include computer storage media and communication media.

Computer storage media can include, without limitation, volatile and nonvolatile media, as well as removable and non-removable media, implemented in any method or technology for storage of information, such as computer-readable instructions, data structures, program modules, or other data. In this regard, computer storage media can include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which can be accessed by the computing device 2002. Computer storage media does not comprise signals per se.

Communication media typically embodies computer-readable instructions, data structures, program modules, or other data in a modulated data signal, such as a carrier wave or other transport mechanism, and can include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also can be included within the scope of computer-readable media.

The computing device 2002 might operate in a network 2006 using logical connections to one or more remote computers 2008. In some aspects, the one or more remote computers 2008 can be located at a variety of locations, such as medical facilities, research environments, and/or clinical laboratories (e.g., molecular diagnostic laboratories), as well as hospitals, other inpatient settings (e.g., surgical centers), veterinary environments, ambulatory settings, medical billing offices, financial offices, hospital administration settings, home healthcare environments, and/or clinicians' offices). As used herein, “clinicians,” “medical professionals,” or “healthcare providers” can include: physicians; specialists such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like.

In aspects, the computing device 2002 uses logical connections to communicate with one or more remote computers 2008 within the computing environment 2000. In aspects where the network 2006 includes a wireless network, the computing device 2002 can employ a modem to establish communications with the Internet, the computing device 2002 can connect to the Internet using Wi-Fi or wireless access points, or the server can use a wireless network adapter to access the Internet. The computing device 2002 engages in two-way communication with any or all of the components and devices illustrated in FIG. 20, using the network 2006. Accordingly, the computing device 2002 can send data to and receive data from the remote computers 2008 over the network 2006.

The network 2006 is a computer network that can include local area networks (LANs) and/or wide area networks (WANs), in some aspects. The network 2006 can include wireless and/or physical (e.g., hardwired) connections. Examples of networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. When the network 2006 includes a WAN-type configuration, the computing device 2002 might comprise a modem or other means for establishing communications over the WAN, such as the Internet, in such aspects. As such, the network 2006, can provide the components and devices access to the Internet and web-based applications.

The network 2006 can include an entity-wide network, campus-wide network, an office-wide network, an enterprise-wide networks, and the Internet. In the network 2006, applications, extensions, program modules or portions thereof might be stored in association with the computing device 2002, the data store 2004, and any of the one or more remote computers 2008. For example, various application programs can reside on the memory associated with any one or more of the remote computers 2008. In the computing environment 2000, which is illustrated as being a distributed configuration of the network 2006, the components and devices can communicate with one another and can be linked to each other using a network 2006. It will be appreciated by those of ordinary skill in the art that the network connections shown are exemplary and other means of establishing a communications link between the computers (e.g., computing device 2002 and remote computers 2008) might be utilized.

In operation, an organization might enter commands and information into the computing device 2002 or convey the commands and information, for example, directly in peer-to-peer or near-field communication, or through the network 2006 using telecommunications or Wi-Fi, to the computing device 2002 via one or more of the remote computers 2008 through input devices, such as a keyboard, a pointing device (e.g., a mouse), a trackball, as stylus, or a touch pad. Other input devices comprise microphones, satellite dishes, scanners, or the like. Commands and information might also be sent directly from a remote healthcare device to the computing device 2002. In addition to a screen, monitor, or touchscreen component, the computing device 2002 and/or remote computers 2008 might comprise other peripheral output devices, such as speakers and printers.

The computing environment 2000 includes one or more remote computers 2008, which may be accessed by the computing device 2002 over the network 2006 or directly using peer-to-peer connections or mesh networking, in various aspects. The remote computers 2008 might be servers, routers, network personal computers, peer devices, network nodes, computing devices, personal digital assistants, personal mobile devices, medical devices, patient monitoring equipment, or the like, and might comprise some or all of the elements described above in relation to the computing device 2002. The one or more remote computers 2008 can include multiple computing devices, in various aspects. In aspects where the network 2006 is distributed in configuration, the one or more remote computers 2008 can be located at one or more different geographic locations. In an aspect where the one or more remote computers 2008 are a plurality of computing devices, each of the plurality of computing devices can be located across various locations such as buildings in a campus, medical and research facilities at a medical complex, offices or “branches” of a banking/credit entity, or can be mobile devices that are wearable or carried by personnel, or attached to vehicles or trackable items in a warehouse, for example. In some aspects, the remote computers 2008 are physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. The remote computers 2008 might also be physically located in nontraditional healthcare environments so that the entire healthcare community might be capable of integration on the network 2006. In other aspects, the remote computers 2008 can be physically located in a non-medical setting, such as a packing and shipping facility or deployed within a fleet of delivery or courier vehicles.

Continuing, the computing environment 2000 includes a data store 2004. Although shown as a single component, the data store 2004 can be implemented using multiple data stores that are communicatively coupled to one another, independent of the geographic or physical location of a memory device. The data store 2004 can, for example, store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. The data store 2004 can, for example, also store data in the form of electronic records, such as electronic medical records of patients, patient-specific documents and historical records, transaction records, billing records, task and workflow records, chronological event records, and the like. Generally, the data store 2004 includes physical memory that is configured to store information encoded in data. For example, the data store 2004 can provide storage for computer-readable instructions, computer-executable instructions, data structures, data arrays, computer programs, applications, and other data that supports the functions and actions to be undertaken using the computing environment 2000 and components shown in the example of FIG. 20.

As shown in the example of FIG. 20, when the computing environment 2000 operates with distributed components that are communicatively coupled via the network 2006, computer instructions, applications, extensions, and/or program modules can be located in local and/or remote computer storage media (e.g., memory storage devices). Aspects of the present invention can be described in the context of computer-executable instructions, such as program modules, being executed by a computing device. Program modules can include, but are not limited to, routines, programs, objects, components, and data structures that perform particular tasks or implement particular abstract data types. In aspects, the computing device 2002 can access, retrieve, communicate, receive, and update information stored in the data store 2004, including program modules. Accordingly, the computing device 2002 can execute, using a processor, computer instructions stored in the data store 2004 in order to perform aspects described herein.

Although internal components of the devices in FIG. 20, such as the computing device 2002, are not illustrated, those of ordinary skill in the art will appreciate that internal components and their interconnection are present in the devices of FIG. 20. Accordingly, additional details concerning the internal construction device are not further disclosed herein. Although many other internal components of the computing device 2002 and the remote computers 2008 are not shown, such components and their interconnection are known. Accordingly, additional details concerning the internal construction of the computing device 2002 and the remote computers 2008 are not further disclosed herein.

Additionally, it will be understood by those of ordinary skill in the art that the computing environment 2000 is just one example of a suitable computing environment and is not intended to limit the scope of use or functionality of the present invention. Similarly, the computing environment 2000 should not be interpreted as imputing any dependency and/or any requirements with regard to each component and combination(s) of components illustrated in FIG. 20. It will be appreciated by those having ordinary skill in the art that the connections illustrated in FIG. 20 are just examples as other methods, hardware, software, and devices for establishing a communications link between the components, devices, systems, and entities can be utilized in implementation of the present invention. Although the connections are depicted using one or more solid lines, it will be understood by those having ordinary skill in the art that the example connections of FIG. 20 can be hardwired or wireless, and can use intermediary components that have been omitted or not included in FIG. 20 for simplicity's sake. As such, the absence of components from FIG. 20 should not be interpreted as limiting the present invention to exclude additional components and combination(s) of components. Moreover, though devices and components are represented in FIG. 20 as singular devices and components, it will be appreciated that some aspects can include a plurality of the devices and components such that FIG. 20 should not be considered as limiting the number of a device or component.

The present invention has now been described in relation to particular aspects, which are intended in all respects to be illustrative rather than restrictive. Thus the present invention is not limited to these aspects, but variations and modifications can be made without departing from the scope of the present invention.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A method facilitating a computerized partogram, the method comprising:
generating a graphical user interface that presents a user-interactive partogram, wherein a plurality of applications are integrated into the user-interactive partogram;
causing display of the user-interactive partogram in the graphical user interface, wherein each of the plurality of applications are displayed in a corresponding portion of the graphical user interface;
receiving user input via the graphical user interface;
modifying, from within the user-interactive partogram, an electronic record in response to the user input; and
updating the user-interactive partogram being displayed in the graphical user interface based on the electronic record being modified.

2. The method of claim 1, wherein the user-interactive partogram is cloud-deployed.

3. The method of claim 2, wherein a representational state transfer (REST) service exposes the plurality of integrated applications that are cloud-deployed to a database that stores the electronic record.

4. The method of claim 3, wherein exposure of the plurality of applications by the REST service enables modifying the electronic record from within the user-interactive partogram.

5. The method of claim 1, wherein the plurality of applications are hosted in an application layer.

6. The method of claim 1, wherein the partogram is hosted in a platform layer.

7. The method of claim 1, wherein the user input comprises a patient-specific value defining systolic blood pressure, diastolic blood pressure, SpO2, maternal heart rate, oral temperature, auxiliary temperature, respiratory rate, or a combination thereof.

8. The method of claim 7, wherein the electronic record is automatically modified by a charting engine to reflect the patient-specific value of the user input, without requiring navigation to a new screen.

9. The method of claim 1, wherein the user-interactive partogram is automatically updated as displayed in the graphical user interface in response to modifying the electronic record and without further user input.

10. One more non-transitory computer-readable media having computer-executable instructions embodied thereon that, when executed, perform a method, the media comprising:
generating a graphical user interface to display a cloud-based user-interactive partogram having a plurality of applications that are integrated into the cloud-based user-interactive partogram, wherein the graphical user interface generated is specific to a first patient;
concurrently displaying the plurality of applications of the cloud-based user-interactive partogram in the graphical user interface, wherein each of the plurality of applications are displayed in a corresponding portion of the graphical user interface, and wherein information specific to the first patient is provided by each the plurality of applications is being updated in near real-time as displayed in the corresponding portion of the graphical user interface;
receiving a request via the graphical user interface;
directly modifying, from within the graphical user interface having the cloud-based user-interactive partogram, an electronic record corresponding to the first patient in response to the request; and
updating the cloud-based user-interactive partogram as displayed in the graphical user interface based on the electronic record being modified.

11. The media of claim 10 further comprising running the cloud-based user-interactive partogram in a cloud-based environment.

12. The media of claim 10 further comprising exposing, by a representational state transfer (REST) service, the plurality of integrated applications to a database that stores a plurality of electronic records.

13. The media of claim 10 further comprising automatically populating each of the plurality of applications of the cloud-based user-interactive partogram with information specific to an individual being monitored.

14. The media of claim 10 further comprising continuously updating the plurality of applications of the cloud-based user-interactive partogram being concurrently displayed in the graphical user interface using near real-time information specific to an individual being monitored.

15. The media of claim 10, wherein the plurality of applications includes a documentation application.

16. The media of claim 15 further comprising enabling, via one or more REST services acting as an intermediary between a database storing the electronic record and the documentation application, direct modification of the electronic record through the documentation application in the cloud-based user-interactive partogram.

17. The media of claim 10, further comprising, for each of a plurality of user input values that is subsequently received via the graphical user interface:
automatically and directly modifying, from within the graphical user interface having the cloud-based user-interactive partogram, the electronic record corresponding to the first patient in response to the input value; and
automatically updating the cloud-based user-interactive partogram as displayed in the graphical user interface based on the electronic record being modified.

18. The media of claim 10 further comprising receiving an input that specifies an onset date and time of labor for a patient.

19. The media of claim 18 further comprising, in response to receiving the onset date and time, automatically retrieving information from the electronic record that is specific to the patient and populating the graphical user interface with the information retrieved.

20. A system comprising:
a cloud-based partogram platform; and
a plurality of applications that are integrated within the cloud-based partogram platform, wherein the plurality of applications include:
a graphical user interface component for generating and causing display of a user-interactive partogram;
a documentation component for direct charting, within the cloud-based partogram platform, information to a patient's electronic record;
an overview component;
a labor curve component;
a contractions component;
a maternal vital signs component;
a maternal labor assessment component;
a medication administration record component;
a fetal heart rate component; and
a fetal assessment component.
